(19) United States
(12) Patent Application Publication
(10) Pub. No.: US 2026/0087846 A1
(43) Pub. Date: Mar. 26, 2026

(54) CAMERA-BASED VITAL FEATURE ESTIMATION

(71) Applicant: THE ROYAL INSTITUTION FOR THE ADVANCEMENT OF LEARNING / MCGILL UNIVERSITY, Montréal (CA)

(72) Inventor: Narges Arman Fard, Montréal (CA)

(21) Appl. No.: 19/340,709

(22) Filed: Sep. 25, 2025

Related U.S. Application Data
(60) Provisional application No. 63/698,600, filed on Sep. 25, 2024.

(51) Int. Cl. G06V 40/10; G06V 10/82; G16H 30/40; G16H 50/70

(52) U.S. Cl. CPC G06V 40/15; G06V 10/82; G16H 30/40; G16H 50/70

(57) ABSTRACT

The present disclosure presents a Multi-Input, Multi-Output artificial intelligence system that jointly processes video with other physiological signals during training to uncover shared representations across modalities and prevent the mislearning observed in existing methods. Once trained, the system requires video input at inference, yet remains capable of reconstructing physiological signals learned during training. By leveraging these crossmodal patterns, the system can use video alone to estimate complex signals such as blood pressure. This integration of video with complementary vital features during training therefore provides a pathway toward accurate, contactless estimation of a broad range of vital signs. A method for estimating vital features of a patient is provided. The method includes receiving a video segment showing a skin-exposed region of the patient, cropping the video segment, and estimating vital features by providing the crops as input to a neural network.

### CROSS-REFERENCE TO RELATED APPLICATIONS

This application claims the benefit of, and priority to, U.S. Provisional Patent Application No. 63/698,600, filed Sep. 25, 2024, and entitled “CAMERA-BASED BLOOD PRESSURE ESTIMATION”, the disclosure of which is hereby incorporated by reference in its entirety.

### TECHNICAL FIELD

The technical field relates to vital feature estimation, and more specifically to multi-input, multi-output systems and methods for estimating the vital features of a patient.

### BACKGROUND

Vital features—including for instance physiological signals such as blood pressure (BP), electrocardiogram (ECG), photoplethysmogram (PPG), and impedance cardiogram (ICG), as well as vital metrics such as heart rate (HR)—are crucial indicators of cardiovascular, respiratory, and systemic health. Accurate estimation of these features is essential for the prevention and management of a wide range of potentially fatal health conditions. Conventional vital feature monitoring techniques commonly involve contact-based devices such as mercury sphygmomanometers. Such methods are unsuitable for continuous or real-time monitoring, which is especially important for timely detection of abnormal fluctuations and trends, aiding in timely intervention to prevent health complications. To address this problem, contactless vital feature monitoring methods have recently emerged.

In particular, studies have shown that blood pressure (BP), photoplethysmography (PPG), electrocardiography (ECG), and impedance cardiography (ICG) signals exhibit correlated patterns that reflect underlying cardiovascular dynamics. The temporal and morphological relationships among these features suggest the presence of a shared physiological signature, making it possible to leverage them jointly. This interdependence provides a strong foundation for developing multi-input, multi-output (MIMO) systems capable of simultaneously predicting multiple cardiovascular metrics by capturing the complementary information embedded across these signals.

In theory, estimating physiological signals from a video is possible because the light illuminating the skin is absorbed differently based on blood volume changes. Moreover, due to the existence of reliable computer vision techniques, a contactless camera-based feature estimation method would be more robust to motion artifacts, which is essential to have in a seamless health monitoring system. Recent studies have demonstrated the successful extraction of PPG, ICG, and even ECG from videos.

However, accurate and reliable estimation of some vital features, such as SBP and DBP, solely through video data has remained unsolved. This is because blood pressure is a complex physiological feature influenced by various factors such as arterial stiffness, vessel diameter, and cardiac output which may not be directly captured from the video without any guidance.

Current technologies, such as the Nuralogix™ Transdermal Optical Imaging™ (TOI) method, face a major challenge in estimating BP from video: SBP and DBP values are typically concentrated around their normal ranges, while clinically meaningful fluctuations occur infrequently. Yet, it is precisely these fluctuations that are of greater interest. Many existing models focus on minimizing overall estimation error; however, in practice, this often results in outputs that default to “normal” SBP/DBP values, which appear accurate in most cases but fail to reflect true physiological variation. Such behaviour does not represent genuine prediction from video, as the underlying blood pressure-related patterns may remain uncaptured. Consequently, reliably extracting and validating these signals from video remains a significant challenge.

### SUMMARY

A multi-input, multi-output system that jointly processes video alongside other physiological signals—such as ECG, PPG, and ICG—can help uncover shared patterns between these modalities. By learning these common patterns, the system can leverage video to estimate challenging signals like blood pressure, even in the absence of other inputs. As earlier discussed, while previous studies have demonstrated that blood pressure can be reliably estimated from physiological signals, reliable estimation directly from video has not yet been achieved. Integrating video with additional vital features thus provides a pathway toward accurate, contactless vital feature estimation.

However, recording data that includes physiological signals while ensuring synchronization between video frames and corresponding signal data points is both challenging and costly, making it difficult in the inference phase of an unobtrusive monitoring system where the goal is to provide continuous measurement. Furthermore, these inputs—video and vital features—are highly heterogeneous and require different processing approaches while still preserving the shared information between them. This is a challenging task that has not yet been addressed.

The multiple-input, multiple-output approach remains largely unexplored by players in this field. By jointly processing multiple signals, it can more effectively capture complex physiological patterns, enabling accurate estimation of challenging vital features such as blood pressure. This approach also enhances robustness in health monitoring and reduces costs compared to maintaining separate single-output systems for each signal, making it a highly promising solution for comprehensive, efficient, and scalable physiological assessment.

There remains a need, therefore, for an integrated framework that allows for performing accurate estimations of vital features from video data.

The present disclosure presents a Multi-Input, Multi-Output artificial intelligence (AI) system that jointly processes video with other physiological signals—such as ECG, PPG, and ICG—during training to uncover shared representations across modalities and prevent the mis-learning observed in existing methods. Once trained, the system requires only a single video input at inference, yet remains capable of reconstructing physiological signals learned during training. By leveraging these cross-modal patterns, the system can use video alone to estimate complex signals such as blood pressure, a task that has not yet been reliably achieved in prior work. This integration of video with complementary vital features during training therefore provides a pathway toward accurate, contactless estimation of a broad range of vital signs.

To address at least some of these challenges, a novel deep framework is provided. The framework employs physiological signals along with the video data in the training phase. The model can take multiple inputs comprising of video and physiological signals and learn the patterns that are shared among them, but can be capable of continuous estimation of vital features solely from video during inference. The described model can include an attention-finding module which identifies regions of interest (ROIs), i.e. important areas in the face for vital feature estimation, and an estimation module, consisting of a multimodal contrastive learning framework, which is used to integrate information from both video and physiological signals.

In some embodiments, three regions of interest are located, which builds on previous cuff-based studies indicating that PTT can be inferred by simultaneously considering multiple human body parts.

In accordance with an aspect, a method for estimating vital signals of a patient is provided. The method includes receiving a video segment showing at least one skin exposed region of the patient (e.g., face), cropping the video segment to create at least one video patch, and generating at least one predicted vital feature value of the patient by providing the at least one video patch as input to a neural network trained on a training dataset including a plurality of training video segments, each training video segment showing at least the one skin exposed region of a training subject and being associated with at least one first time series corresponding to values of a measured physiological signal of the training subject. The neural network is configured to generate a representation of at least one-time series of predicted values of a predicted physiological signal of the patient.

In some embodiments, the neural network includes a neural network-based encoder trained to accept the at least one video patch as input and to generate a first vectorial embedding as output, at least one fully connected block trained to accept the first vectorial embedding as input and to generate at least one second vectorial embedding as output corresponding to the representation of the time series of predicted values of the predicted physiological signal of the patient, and at least one fully connected block trained to accept an aggregation of the at least one second vectorial embedding and to generate the at least one predicted vital feature value.

In some embodiments, cropping the video segment includes using an attention module trained using reinforcement learning based on a reward corresponding to an error of the neural network in predicting the measured values of the blood pressure of the training subject.

In accordance with an aspect, a method for estimating at least one vital feature of a patient is provided. The method includes receiving a video segment showing at least one skin exposed region of the patient, cropping the video segment to create at least one video patch, and generating at least one predicted vital feature of the patient by providing the at least one video patch as input to a neural network trained on a training dataset including a plurality of training video segments, each training video segment showing at least one skin exposed region (e.g., face) of a training subject and being associated with at least one first time series corresponding to values of a measured physiological signal of the training subject and at least one second time series or vital metrics corresponding to measured values of the vital feature of the training subject. A time alignment block is configured to synchronize and temporally align the video, time series, and vital metrics. The neural network is configured to generate a representation of at least one time series of predicted values of a predicted physiological signal of the patient and to generate the at least one predicted vital feature value based on the representation.

In some embodiments, a system for estimating multiple vital features of a patient from a video segment showing at least one skin-exposed region of the patient.

In some embodiments, the neural network includes an encoder trained to accept the at least one video patch as input and to generate a first vectorial embedding as output, at least one fully connected block trained to accept the first vectorial embedding as input and to generate at least one second vectorial embedding as output corresponding to the representation of the time series of predicted values of the predicted physiological signal of the patient, and at least one fully connected block trained to accept an aggregation of the at least one second vectorial embedding and to generate the at least one predicted vital feature value.

In some embodiments, cropping the video segment includes using an attention module trained using reinforcement learning based on a reward corresponding to an error of the neural network in predicting the measured values of the vital features of the training subject.

In accordance with an aspect, a system for estimating vital features of a patient from a video segment showing at least one skin-exposed region of the patient is provided. The system includes at least one memory for storing parameters of a neural network. The neural network includes an encoder trained to accept at least one video patch of the video segment as input and to generate a first vectorial embedding as output, at least one fully connected block trained to accept the first vectorial embedding as input and to generate at least one second vectorial embedding as output corresponding to a representation of a time series of predicted values of a physiological signal of the patient, and at least one fully connected block trained to accept an aggregation of the at least one second vectorial embedding and to generate at least one predicted blood pressure value for the patient.

In another embodiment, the system incorporates additional encoders beyond the RGB video encoder, each corresponding to a specific input modality (e.g., vital signals, metrics, and/or thermal or infrared (IR) video). These encoders are trained to process at least one segment of their respective modality and generate vector embeddings as output. A multimodal block is then trained to integrate these embeddings and produce synchronized secondary embeddings that represent shared physiological patterns of the patient. Finally, a fully connected block aggregates the secondary embeddings to generate one or more predicted vital feature values.

In some embodiments, two or more of the encoders may be merged.

In some embodiments, the at least one memory further stores parameters of an attention module trained to crop the at least one video patch from the video segment.

In some embodiments, the model outputs multiple vital features as output.

In some embodiments, the input video is from RGB cameras.

In some embodiments, the input video is from infrared cameras.

In some embodiments, the input video is from both RGB cameras and infrared cameras.

In some embodiments, the model could be deployed on edge devices and output real-time prediction.

In some embodiments, the model is zero-shot and can predict the vital signals of a new patient without calibration.

In accordance with an aspect, a computer-implemented method for estimating at least one vital feature of a patient is provided. The method includes receiving a video segment showing at least one skin-exposed region of the patient, cropping the video segment to create at least one video patch, and generating the at least one estimated vital feature of the patient by providing the at least one video patch as input to a neural network trained on a training dataset comprising a plurality of training video segments of a plurality of training subjects, each training video segment showing at least one skin-exposed region of a training subject of the plurality of training subjects and being associated with at least one first time series corresponding to values of a measured physiological signal of the training subject and at least one second time series corresponding to measured values of a vital feature of the training subject, wherein the neural network is configured to generate a representation of at least one-time series of predicted values of a predicted physiological signal of the patient and to generate the at least one estimated vital feature based on the representation, and wherein the patient and the plurality of training subjects belong to different populations.

In accordance with an aspect, a system for estimating at least one vital feature of a patient from at least a video segment showing at least one skin-exposed region of the patient is provided. The system includes at least one memory for storing parameters of a neural network, the neural network comprising: an encoder trained to accept at least one video patch of the video segment as input and to generate a first vectorial embedding as output, at least one fully connected block trained to accept at least the first vectorial embedding as input and to generate at least one second vectorial embedding as output corresponding to a representation of a time series of predicted values of a physiological signal of the patient, and at least one fully connected block trained to accept an aggregation of the at least one second vectorial embedding and to generate the at least one estimated vital feature for the patient.

In accordance with an aspect, a non-transitory computer-readable medium is provided. The computer-readable medium has instructions stored thereon which, when executed by one or more processors, cause the one or more processors to receiving a video segment showing at least one skin-exposed region of a patient, cropping the video segment to create at least one video patch, and generating at least one estimated vital feature of the patient by providing the at least one video patch as input to a neural network trained on a training dataset comprising a plurality of training video segments of a plurality of training subjects, each training video segment showing at least one skin-exposed region of a training subject of the plurality of training subjects and being associated with at least one first time series corresponding to values of a measured physiological signal of the training subject and at least one second time series corresponding to measured values of a vital feature of the training subject, wherein the neural network is configured to generate a representation of at least one-time series of predicted values of a predicted physiological signal of the patient and to generate the at least one estimated vital feature based on the representation, and wherein the patient and the plurality of training subjects belong to different populations.

### BRIEF DESCRIPTION OF DRAWINGS

For a better understanding of the embodiments described herein and to show more clearly how they may be carried into effect, reference will now be made, by way of example only, to the accompanying drawings which show at least one exemplary embodiment.

FIG. 1A is a high-level schematic of a system for training a model to estimate vital features, in accordance with an embodiment.

FIG. 1B is a high-level schematic of a system for using a model to estimate vital features, in accordance with an embodiment.

FIG. 2 is a schematic of a system for estimating SBP and DBP values, as two vital features that could be estimated, in accordance with an embodiment.

FIGS. 3A and 3B are illustrations of a step for training an attention module, in accordance with an embodiment.

FIG. 4A is a flowchart of a method for training a model to estimate two vital features, e.g., SBP, and DBP, in accordance with an embodiment.

FIG. 4B is a flowchart of a method for estimating two vital features, e.g., SBP and DBP values, using a trained model, in accordance with an embodiment.

FIG. 5 is a table presenting performance metrics of a model to estimate two vital features, e.g., SBP, and DBP values, as disclosed herein and of baseline models, in accordance with an experiment performed using an embodiment.

FIGS. 6A, 6B, 6C, and 6D are illustrations of the histogram of a model's error to estimate two vital features, i.e., SBP and DBP values, as disclosed herein and of a baseline model, in accordance with an experiment performed using an embodiment.

FIG. 7 is a table presenting performance metrics of models to estimate two vital features, i.e., SBP and DBP values, as disclosed herein, in accordance with an experiment performed using various embodiments.

FIG. 8 is an illustration of regions of interests identified by attention module as disclosed herein, in accordance with an experiment performed using an embodiment.

FIG. 9 is a table presenting performance metrics of models to estimate two vital features, SBP and DBP values, as disclosed herein, in accordance with an experiment performed using various embodiments.

FIG. 10 is a table presenting performance metrics of models to estimate two vital features, SBP and DBP values, as disclosed herein and of baseline models, in accordance with an experiment performed using various embodiments.

### DETAILED DESCRIPTION

It will be appreciated that, for simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practised without these specific details. In other instances, well-known methods, procedures and components have not been described in detail so as not to obscure the embodiments described herein. Furthermore, this description is not to be considered as limiting the scope of the embodiments described herein in any way but rather as merely describing the implementation of the various embodiments described herein.

One or more methods and systems described herein may be implemented in computer program(s) executed on processing device(s), each comprising at least one processor, a data storage system (including volatile and/or non-volatile memory and/or storage elements), and optionally at least one input and/or output device. “Processing devices” encompass computers, servers and/or specialized electronic devices which receive, process and/or transmit data. As an example, “processing devices” can include processing means, such as microcontrollers, microprocessors, and/or CPUs, or be implemented on FPGAs. For example, and without limitation, a processing device may be a programmable logic unit, a mainframe computer, a server, a personal computer, a cloud-based program or system, a laptop, a personal data assistant, a cellular telephone, a smartphone, a wearable device, a tablet, a video game console or a portable video game device.

Each program is preferably implemented in a high-level programming and/or scripting language, for instance an imperative e.g., procedural or object-oriented, or a declarative e.g., functional or logic, language, to communicate with a computer system. However, a program can be implemented in assembly or machine language if desired. In any case, the language may be a compiled or an interpreted language. Each such computer program is preferably stored on a storage media or a device readable by a general or special purpose programmable computer for configuring and operating the computer when the storage media or device is read by the computer to perform the procedures described herein. In some embodiments, the system may be embedded within an operating system running on the programmable computer.

Furthermore, the system, processes and methods of the described embodiments are capable of being distributed in a computer program product comprising a computer readable medium that bears computer-usable instructions for one or more processors. The computer-usable instructions may also be in various forms including compiled and non-compiled code.

The processor(s) are used in combination with storage medium, also referred to as “memory” or “storage means”. Storage medium can store instructions, algorithms, rules and/or trading data to be processed. Storage medium encompasses volatile or non-volatile/persistent memory, such as registers, cache, RAM, flash memory, ROM, diskettes, compact disks, tapes, chips, as examples only. The type of memory is, of course, chosen according to the desired use, whether it should retain instructions, or temporarily store, retain or update data. Steps of the proposed method are implemented as software instructions and algorithms, stored in computer memory and executed by processors.

The proposed technology is capable of extracting vital features include BP, ECG, PPG, ICG, body temperature, HR, heart rate variability (HRV), and respiratory rate (RR, obtained via different encoders), blood oxygen level, along with the P-QRS-T components of the ECG waveform from distant video.

The vital features considered in this disclosure provide a comprehensive view of systemic health by capturing both cardiovascular and broader physiological states. The blood pressure (BP) signal, heart rate (HR), and electrocardiogram (ECG) provide direct insight into cardiac function, rhythm, and vascular status.

The P-QRS-T components of the ECG allow for a detailed assessment of the electrical activity of the heart. The photoplethysmogram (PPG) and impedance cardiogram (ICG) complement these measures by providing information about blood volume changes, cardiac output, and arterial compliance. Heart rate variability (HRV) and respiratory rate (RR) reflect autonomic nervous system function and overall cardiorespiratory regulation. Body temperature captures metabolic and thermoregulatory status. Blood pressure has prominent components, namely systolic blood pressure (SBP) and diastolic blood pressure (DBP). SBP represents the pressure in the arteries when the heart contracts and pumps blood, whereas DBP is the pressure when the heart is at rest between beats. Both SBP and DBP values are essential indicators of cardiovascular health and are useful for assessing an individual's overall physiological well-being. Together, these multimodal signals enable continuous, noninvasive monitoring of systemic physiological health, supporting early detection of cardiovascular, respiratory, and autonomic dysfunction, as well as providing valuable metrics for general wellness assessment as discussed for instance in Pastor-Barriuso, R., et al.: Systolic blood pressure, diastolic blood pressure, and pulse pressure: An evaluation of their joint effect on mortality. Annals of Internal Medicine 139(9), 731-739 (2003), the disclosure of which is hereby incorporated by reference in its entirety.

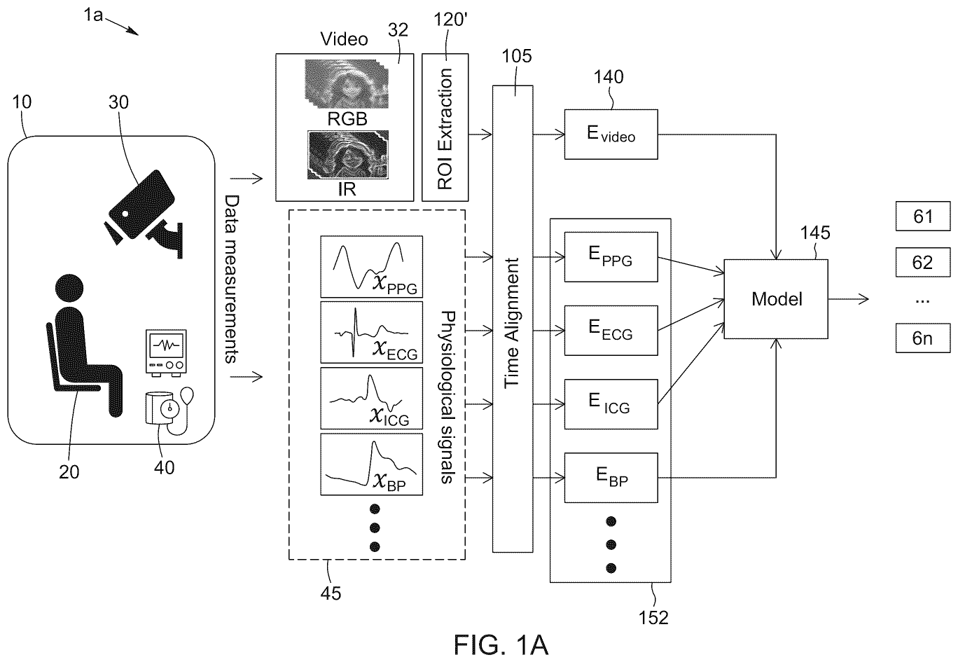

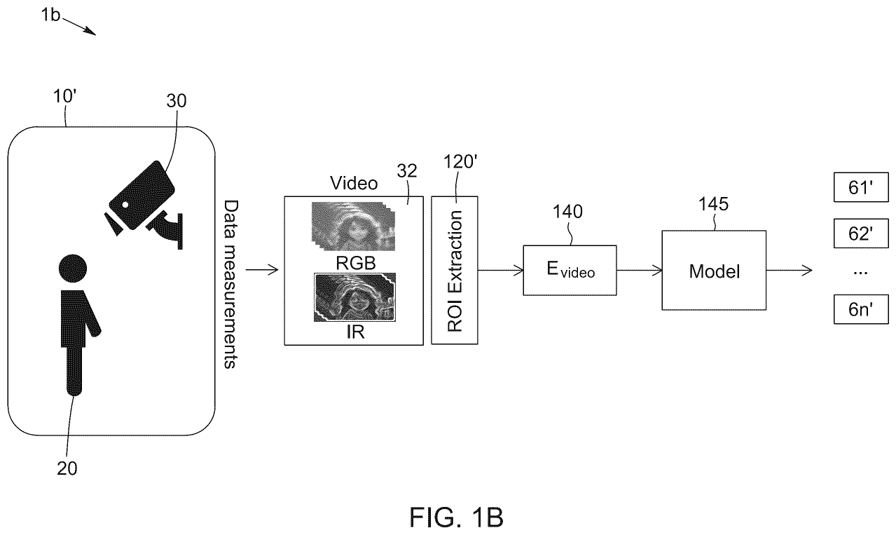

With reference to FIG. 1A, an exemplary system 1a for training a model portion 145 to estimate, or predict one or more vital feature value such as SBP and DBP values from T-frame video recordings of subjects is shown. The time span of the T frames can be as small as one heartbeat but preferably is not too large to miss potential abrupt feature variations, which can be important for health monitoring purposes.

In some embodiment, system 1a corresponds to a multimodal input and multi-feature output system for training a model to estimate n vital features 61, 62, . . . , 6n. In other words, the system 1a can take multiple vital modalities, such as blood pressure (denoted by x_{BP}), as input, align them, and feed them into one or more encoders 140, 152 (denoted by E in the figure), each corresponding to an input modality, to extract embeddings. The encoders can for instance be neural encoders. Some or all of the encoders can be autoencoders, i.e., encoders trained using unsupervised learning, and some or all of the encoders can be trained along with the model portion 145 in an end-to-end manner. In some embodiments, one or more of the encoders can be pre-trained on a large corpus of data and subsequently fine-tuned jointly with the model portion 145. The neural network can be configured during training to learn a shared representation capable of predicting the vital features with a low estimation error.

System 1a includes a data measurement apparatus 10 for acquiring data related to a training subject 20. The apparatus 10 includes a video camera 30 configured to create video segments 32 that show at least a skin-exposed region of the subject 20, e.g., the face of the subject 20. In some embodiments, the video camera 30 is an RGB video camera configured to create colour video segments. In some embodiments, the video camera 30 is a thermal and/or infrared camera configured to create thermal images of the subject. In some embodiments, the video camera 30 corresponds to multiple cameras and/or include multiple sensors, for instance an RGB sensor to generate video segments and an IR sensor to generate thermal images. The apparatus 10 also includes medical equipment 40 configured to acquire physiological information, including for instance values of one or more physiological signals, e.g., photoplethysmography (PPG), electrocardiography (ECG), and impedance cardiogram (ICG) signals, and values of one or more vital feature values, e.g., systolic blood pressure (SBP) and diastolic blood pressure (DBP), of the subject 20. The values acquired by the medical equipment 40 can be arranged as time series synchronized with the frames of video segments 32, thereby creating at least one first time series 45 each corresponding to values of a measured physiological signal of the training subject 20 and at least one second time series corresponding to measured values of a target vital signal, such as blood pressure, of the training subject 20.

Let Xᵥ∈ [symbol] ^{T×3×H×W} be the input video 32 in which T is the number of frames within the video segment, H and W respectively denote the video frame height and width, and 3 refers to the three red, green and blue (RGB) channels of the input video. The goal of system 1a is to learn a mapping for the target vital features, such as blood pressure 60 approximation, which takes Xᵥ as input and outputs SBP and/or DBP, which are two real numbers. In some embodiments, an region-of-interest (ROI) process (120′) takes place and the extracted patches are used as input to infer the target vital features.

During training, which can also be referred to as the calibration phase, a dataset of physiological signals, xₛ∈ [symbol] ^{1×T} where s∈{PPG, ECG, ICG}, can be used. In some embodiments, xₛ are recorded synchronously with Xᵥ meaning the frame associated with each physiological data sample can be uniquely identified. The objective is to train the model 100 or model portion 145 so that it can capture relevant patterns for estimating SBP/DBP in a short T-Frame video recording. As an example, T can be set to approximately 50, corresponding to an approximately 2-second video segment at 24 fps. In some embodiments, the video 32 and/or video patches and the measured physiological signals 45 undergo a time alignment process 105. In some embodiments, the video 32 and/or video patches are sent to a video encoder 140 and the measured physiological signals 45 are each sent to a corresponding encoder 152 to obtain, for each of the video and the measured physiological signals, a vectorial embedding that is used to train a portion of the model 145.

With reference to FIG. 1B, an exemplary system 1b for using a model portion 145 to estimate, or predict one or more vital feature value such as SBP and DBP values from T-frame video recordings of subjects is shown. In some embodiments, system 1b corresponds to a multimodal input and multi-feature output system for using a model to estimate n vital features 61′, 62′, . . . , 6n′.

System 1b includes a data measurement apparatus 10′ for acquiring data related to an individual, e.g., a patient 20′. The apparatus 10′ includes a video camera 30 configured to create a video segment 32 that show at least a skin exposed region such as the face of the patient 20. As with system 1a shown in FIG. 1A, video camera 30 can include for instance RGB and/or IR sensors. The video segment 32 can be input in the model 100, or an embedding corresponding to a representation of video segment 32 can be input in the model portion 145, to obtain one or more predicted vital feature values 61′, 62′, . . . , 6n′.

In some embodiments, the video segment 32 undergoes a ROI extraction process 120′. In some embodiments, the video segment 32 and/or the ROIs are passed as input to a video encoder 140 to generate a vectorial embedding that is then passed as input to a remaining portion of the model 145.

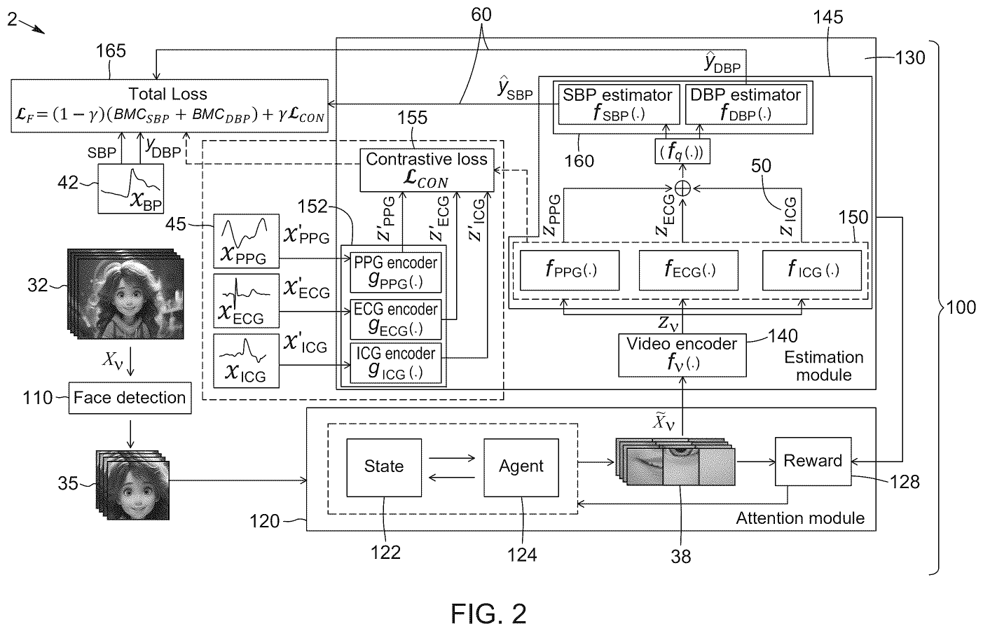

With reference to FIG. 2, an exemplary system 2 for estimating one or more target vital features, e.g., blood pressure, SBP and/or DBP parameters is shown. Broadly described, system 2 includes an attention module 120 based on a reinforcement learning framework that crops the videos 32, i.e., decides which patches 38 of the skin exposed region per frame, i.e., ROIs, will be used for BP estimation of a specific subject, and an estimation module 130 which includes encoder functions that map video of the selected patches 38 and the physiological signals 45 into embedding vectors, along with a multimodal contrastive learning module which aims to align these representations with each other. The estimation module 130 can output the estimated vital feature 60 and can also be used for reward generation required for the attention module 120.

In some embodiments, a face detection module 110 is further used to detect the face of a subject from a video segment 32 showing more than just the face and perform a preliminary cropping 35 of the video segment 32, to simplify the work of the attention module 120 and such that processing of the video segment 32 by the attention module 120 requires fewer computational resources.

The attention module 120 and the estimation module 130 can together be referred to as the “model” 100 in the present disclosure. In embodiments using a face detection module 110, the face detection module 110 can also be referred to as being part of the model 100. In some embodiments, the attention module 120 can be absent from system 2, such that the estimation module 130 is trained to input directly video segments 32 or cropped video segments 35. Nonetheless, as is shown further down below, systems using an attention module 120 working in cooperation with an estimation module 130 trained to input video patches 38 from the attention module 120 provide improved performances.

In some embodiments, the model 100 includes an attention module 120 configured to crop video segments 32 or 35 and to output a configurable number of patches 38 corresponding to “attentive areas” of the video, of a configurable size, or of a size determined based on the resolution of the video segments 32 or 35. It can be appreciated that a higher number of patches 38 provide more accurate results. As an example, using one patch 38 does not provide very accurate results. As another example, using three patches 38 provide very accurate results.

Advantageously, using two patches 38 provide good results while using less computational resources such as memory and processor time than using more patches, representing an advantageous performance-resource usage tradeoff. The task of identifying the attentive areas in video frames can be modelled as a Markov decision process, and can employ the REINFORCE algorithm, a popular policy-based reinforcement learning method based on the Monte Carlo policy gradient approach. The agent 124 performs actions A in its current state 122 S, which leads to changing its state and getting a reward 128 R. The agent 124 learns to select the relevant areas in each frame, i.e., spatial hard attention, by maximizing the total expected reward 128. In the present disclosure, the k^{th} step of the RL episode is denoted by Tₖ=(Sₖ, Aₖ, Rₖ), where Sₖ, Aₖ, and Rₖ are state, action, and reward at the k^{th} step. Therefore, one single episode is denoted as T=(S₁, A₁, R₁, . . . , S_{K}, A_{K}, R_{K}).

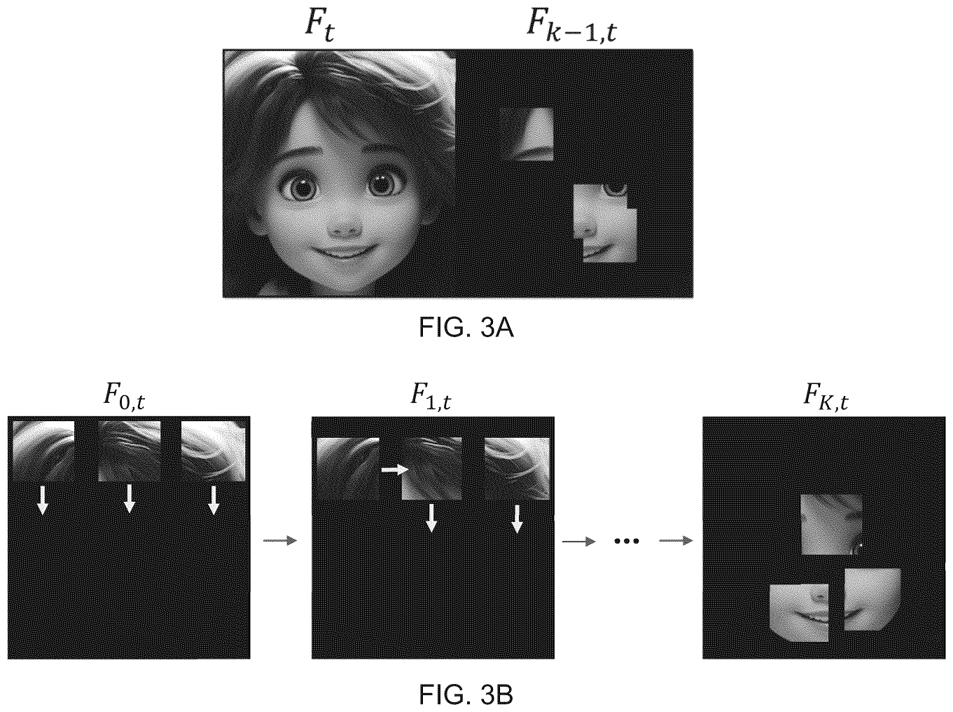

The states 122 can be defined as follows. As the previous studies have shown, as discussed for instance in Qin, K., Huang, W., Zhang, T., Tang, S., op. cit., face is the most informative region for extracting physiological information; therefore, considering the existence of strong face detector techniques and in order to reduce the search space of the agent, avoid large computations, and improve the robustness and reliability of the method. Therefore, in some embodiments, the face Fₜ is first identified by a face detection module 110 in each frame, and then, uses in state definition. There are several well-established face detection frameworks that can achieve near-perfect accuracy, such as Haar Cascade Classifier, which is a popular face detector. The state S_{k,t} is defined by concatenating Fₜ and the output of the agent 124, i.e., the selected patches 38, from the previous step of the episode denoted as Fₖ₋₁, i.e., S_{k,t}=[Fₜ, F_{k-1,t}]. It can be noted that Fₜ is the face area in the t^{th} frame, which has a different size from the original frame. F_{k-1,t} has the same size as Fₜ, where only the selected patches appear within the frame, while all other areas are assigned a value of zero. An example of F_{k-1,t} is shown in the right side of FIG. 3A. In the initial state, denoted as S^{0,t}, F_{0,t} is initiated such that the patches 38 are located in an arbitrary location such as image corners. As an example, in embodiments with three patches, the initial patches can be located at the top of the image, similar to what is shown in FIG. 3B.

The agent 124 can be defined as follows. A ResNet50 convolutional neural network (CNN) can be used. At the k^{th} t [symbol] ^{th} frame, the agent S_{k,t} take the state P_{k,t} as input and outputs the probability matrix P_{k,t}, describing the actions it can accomplish.

The action can be defined as follows. The action defined for the agent 124 shows the direction of the movements of the patches in the episode. Hence, five actions can be designated: go up, down, left, right, and apply no change. The movement step size can be set to b. For the t^{th} frame, each action in set Aₖ is denoted by

$${a}_{k , t}^{ξ}$$

where k refers to the k^{th} step of the RL episode and ξ is the number of the patch, e.g., 1 for the first patch, 2 for the second patch, and 3 is for the third patch. The actions are sampled from a categorical distribution formed based on P_{k,t}. FIG. 3B illustrates an example of one episode.

The reward 128 can be defined as follows. The reward can correspond to the effectiveness of the agent's actions in reaching its goal. In system 2, the goal is to improve the vital feature estimation. Consequently, the estimation module 130 can be used to evaluate the agent's output in step k of the episode. To do so, a sequence of T frames in the video segment can be created with the selected n×n patches in the frames, and the three newly created video segments (each with size T×n×n) can be concatenated next to each other; then fed to the estimation module 130 to get the desired vital feature, which is systolic blood pressure and diastolic blood pressure values in this case 60. Note that, for the T frames, the estimation module 130 can be configured to output two scalar values, one for SBP and one for DBP. Then, the balanced mean square errors,

$${MSE}_{SBP}^{k} , {MSE}_{DBP}^{k} ,$$

which are obtained by comparing the predicted SBP and DBP in the k^{th} step with their true values, can be calculated and the agent's reward in the k^{th} step can be defined as:

$${R}_{k} = - \frac{1}{2} ( {MSE}_{SBP}^{k} + {MSE}_{DBP}^{k} ) .$$

In some embodiments, the attention module 120 is trained with REINFORCE. The objective of the reinforcement learning agent is to acquire a policy function, which identifies the important locations of attentive areas, by maximizing the expected rewards R(θ):

$$ℛ ( θ ) = {𝔼}_{{p}_{θ} ( ( {α}_{k , t}^{ξ} )} [ {R}_{k} ] ,$$

where

$${p}_{θ} ( {α}_{k , t}^{ξ} )$$

is the probability distribution of the possible actions with k={1, . . . , K}, t={1, . . . , T} and ξ={1, 2, 3}(in embodiments with three patches), and [symbol] shows the expected value operator.

In some embodiments, the attention module 120 is configured to ensure that patches 38 do not overlap. As examples only, this feature can be implemented by the agents 124 and/or as part of the reward 128, or as hard-coded restrictions that can be external to the training process.

The REINFORCE method, which is a popular policy gradient algorithm, can be employed to find the optimal parameter 0, which is used to approximate the policy function. Based on REINFORCE, the gradient of the expected reward with respect to the parameter 0, in the k^{th} step of the episode can be calculated as:

$${∇}_{θ} ℛ ( θ ) = {𝔼}_{{p}_{θ} ( {α}_{k , t}^{ξ} )} [ {R}_{k} {∇}_{θ} ln {π}_{θ} ( {α}_{k , t}^{ξ} \vert {S}_{k , t} ) ,$$

where π_{θ} indicates the policy function, and S_{k,t} is the state of the t^{th} frame in the video segment at the k^{th} step

$${α}_{k , t}^{ξ}$$

is the action in the t^{th} frame, and in the k^{th} step of the episode, for the n^{th} region. As previously stated, during each episode, the agent 124 takes K steps for each frame of the video segment 32 or 35. Therefore, the average gradient can be computed over the T frames. In some embodiments, to improve convergence and reduce variance while training the parameter 0, the reward is normalized by deducting a constant baseline value c. The baseline c can for instance be equivalent to the mean reward across episodes. Therefore, the resulting gradient would correspond to:

$${∇}_{θ} ℛ ( θ ) \approx \frac{1}{KT} {\sum }_{k = 1}^{K} [ ( {R}_{k} - c ) {\sum }_{t = 1}^{T} {\sum }_{ξ = 1}^{3} {∇}_{θ} ln {π}_{θ} ( {α}_{k , t}^{ξ} \vert {S}_{k , t} ) .$$

Hence, the policy loss to minimize can be expressed as:

$${ℒ}_{ATT} = \frac{1}{KT} {\sum }_{k = 1}^{K} [ ( {R}_{k} - c ) {\sum }_{t = 1}^{T} {\sum }_{ξ = 1}^{3} {∇}_{θ} ln {π}_{θ} ( {α}_{k , t}^{ξ} \vert {S}_{k , t} ) .$$

The model 100 includes an estimation module 130, configured to generate at least one predicted vital feature value 60, either for a training subject during the training stage, or for a patient when used during inference. In some embodiments, estimation module 130 is also configured to cause the attention module 120 to receive a suitable reward 128 during the training stage. Broadly described, the estimation module 130 can include one or more encoders 140, 152, providing embedding representation of video and/or physiological signals to an estimation portion of the model 145. The model portion 145 can itself include one or more physiological signal estimation blocks 150 feeding one or more vital feature estimation blocks 160.

The estimation module 130 includes a convolutional video encoder 140 trained as a spatio-temp oral video encoder to accept at least one video patch 38 of the video segment 32 and to generate a vectorial embedding as output. In embodiments with an attention module 120, the video encoder 140 is configured to use the output of the RL agent from the attention module 120, extract the selected ROIs per frame and concatenate them next to each other to build \tilde{X}ᵥ. It can be appreciated that using patches 38 rather than the entire frames of video segments 32 causes the video encoder 140 to have a smaller input size, and therefore to use less computational resources such as memory and/or processor time. The video encoder 140 can be described as a function ƒ_{v(⋅)}, which maps s∈{PPG, ECG, ICG} into an embedding vector zᵥ. Then, blocks 150, including for instance one independent fully connected (FC) block for each physiological signal available at training time, each denoted by ƒₛ(.)∈ [symbol] ^{d}, where s∈{PPG, ECG, ICG}, maps zᵥ, into latent embeddings zₛ∈ [symbol] ^{d}, where d is the dimension of each of zₛ. As an example, using PPG, ECG and ICG as physiological signals: zᵥ=ƒᵥ( [symbol] ); zₛ=ƒₛ(zᵥ), s∈{PPG, ECG, ICG}.

Using SBP and DBP as predicted vital feature values, the final predictions ŷ_{SBP} and ŷ_{DBP} are obtained by concatenating the signals passing it through a multilayer perceptron ƒ_{q}(⋅) and two final vital signal estimator blocks 160 ƒ_{SBP}(⋅) and ƒ_{DBP}(⋅), for instance each comprising one linear layer: ŷ_{SBP}=ƒ_{SBP}(ƒ_{q}(z_{PPG}⊕z_{ECG}⊕z_{ICG})), ŷ_{DBP}=ƒ_{DBP}(ƒ_{q}(z_{PPG}⊕z_{ECG}⊕z_{ICG})), where ⊕ is the concatenation operation.

In other words, it could be said that a general representation from video frames is first extracted using ƒᵥ(.), and then patterns that are relevant to each of the physiological signals are extracted using ƒₛ(.). Finally, the networks ƒ_{SBP}(.) and ƒ_{DBP}(.) take the concatenated information and estimate the vital feature values, e.g., the systolic and diastolic blood pressure.

In some embodiments, Video ResNet is used as the backbone of video encoder 140 ƒᵥ. Video ResNet, also known as 3-Dimensional (3D) ResNet, is an extension of the popular ResNet architecture for video analysis tasks. ResNet is renowned for its ability to train very deep neural networks effectively by introducing residual connections. Video ResNet leverages (2+1)D convolutions, combining spatial 2D convolutions for individual frames and 1D convolutions for temporal relationships across video frames. This enables the network 140 to capture spatio-temp oral features effectively. Residual connections are used to facilitate the training of deep networks, allowing Video ResNet to learn hierarchical representations of both spatial and temporal patterns.

In some embodiments, during the training phase, the estimation module 130 is configured to encode the physiological signals time series 45 of the training subjects. One independent encoder 152 can be used for each physiological signal. As an example using physiological signals PPG, ECG and ICG, each encoder 152 can be designated as gₛ(.) with s∈{PPG, ECG, ICG}, to map the features of PPG, ECG, and ICG signals into their corresponding embedding vectors. In some embodiments, since the raw bio-signals are non-stationary and commonly noisy, l statistical and/or temporal features are first advantageously extracted. Features can for instance include the peak value, peak time, variance, and mean of the signal. The resulting feature vectors

$${x}_{s}^{′}$$

with s∈{PPG, ECG, ICG} can then be used as input to the encoders 152. The encoders map the l dimensional

$${x}_{s}^{′}$$

into new representations:

$${z}_{s}^{′} = {g}_{s} ( {x}_{s}^{′} ) ,$$

s∈{PPG, ECG, ICG}.

In some embodiments, multimodal-learning techniques are used to learn a joint representation from video and physiological signals. As an example, multimodal contrastive learning can be used. Contrastive learning is a type of self-supervised learning where the model is trained to pull similar data samples, i.e., positive pairs, closer together in the learned feature space and push dissimilar samples, i.e., negative pairs, further apart. This can be achieved by using a contrastive loss function 155. In the context of the present disclosure, multimodal contrastive learning can mean that the model 100 learns to align videos 32 and physiological signals 45 in the same shared feature space. It can be achieved using a contrastive loss 155 that compares positive pairs (a video and associated signals) against negative pairs (a video and signals of different segments).

Let Pᵥ and Pₛ denote video and physiological data distribution, and

$$( {\tilde{X}}_{v} , {x}_{s}^{′} )$$

indicate paired data from video and physiological signals. The video-signal pair coming from the same time segment can be defined as the positive pair. Independent samples from different segments of each modality can be drawn,

$${\tilde{X}}_{v}^{-} ∼ {P}_{v} , {x}_{s}^{′ -} ∼ {P}_{p}$$

to generate

$$( {\tilde{X}}_{v} , {x}_{s}^{′ -} ) and ( {\tilde{X}}_{v}^{-} , {x}_{s}^{′} )$$

as negative pairs. The contrastive loss 155 then encourages the model 100 to maximize the similarity between the representations of positive pairs while minimizing the similarity between those of negative pairs.

This way, the model 100 learns to associate video 32 with their correct physiological signals 45 and vice versa, effectively learning a mutual understanding of video and signals. To this end, the symmetric cross entropy (SCE) can be used as the contrastive loss function 155:

$${ℒ}_{SCE} ( {\tilde{X}}_{v} , {x}_{s}^{′} ) = - {𝔼}_{{\tilde{X}}_{v} , {x}_{s}^{′}} log \frac{{e}^{{{f}_{s} ( {f}_{v} ( {\tilde{X}}_{v} ) )}^{T} {g}_{s} ( {x}_{s}^{′} )}}{{𝔼}_{{x}_{s}^{′}} - {e}^{{{f}_{s} ( {f}_{v} ( {\tilde{X}}_{v} ) )}^{T} {g}_{s} ( {x}_{\overline{s}}^{′} )}} - {𝔼}_{{\tilde{X}}_{v} , {x}_{s}^{′}} log \frac{{e}^{{{f}_{s} ( {f}_{v} ( {\tilde{X}}_{v} ) )}^{T} {g}_{p} ( {x}_{s}^{′} )}}{{𝔼}_{{\tilde{X}}_{v}} - {e}^{{{f}_{s} ( {f}_{v} ( {\tilde{X}}_{\overline{v}} ) )}^{T} {g}_{s} ( {x}_{s}^{′} )}} ,$$

where [symbol] is the expected value operator. This loss function pulls positive pairs together in the latent space while pushing apart negative pairs.

In some embodiments, the whole network 130 can be trained in an end-to-end manner by adding the weighted loss functions for each branch. As an example, using PPG, ECG and ICG as physiological signals:

$${ℒ}_{CON} = {λ}_{1} {L}_{SCE} ( , {x}_{PPG}^{′} ) + {λ}_{2} {L}_{SCE} ( , {x}_{ECG}^{′} ) + ( 1 - {λ}_{1} - {λ}_{2} ) {L}_{SCE} ( , {x}_{ICG}^{′} ) ,$$

where 0≤λ1, λ2≤1 balance the contribution of each of the networks in the overall loss.

In some embodiments, the overall loss function 165 of the estimation module 130 is the sum of the Batch-based Monte-Carlo (BMC) errors of the predictions, e.g., SBP and DBP predictions, and [symbol] _{CON} using a balancing hyperparameter γ as: [symbol] _{F}=(1−γ)(BMC_{SBP}+BMC_{DBP})+γ [symbol] _{CON}, where the ground-truth SBP and DBP are used to calculate BMC errors.

In some embodiments, the attention module 120 and the estimation module 130 are trained jointly. This ensures that both the loss L_{ATT} of the attention module and the loss L_{F} of the estimation module 130 are minimized, which ensures comprehensive training of all components in the model. However, simultaneously updating all the parameters could lead to instability in loss reduction due to the inherent noise and complexity of each task. Moreover, the estimation module 130 can be involved in the training process of the attention module 120 by providing rewards; hence, adapting the estimation module 130 to the output of the attention module 120 helps improve the overall performance of the model 100. To address these challenges, a two-step training strategy can be adopted. In the first step, a fraction K of the training data can be selected and used to minimize L_{F} while keeping the selected attentive regions fixed for a predefined number of epochs. This targeted optimization allows for a more stable reduction in the contrastive loss 155. In the second step, the remaining data portion (1−κ) can be used to update the location of the attentive regions through minimizing L_{RL} for a predefined number of epochs, for instance for the same number of epochs. This sequential approach can be repeated until a condition is attained, for instance after finishing a predefined number of epochs. This approach can help to navigate the complex training dynamics, ensuring that both losses are minimized without compromising stability.

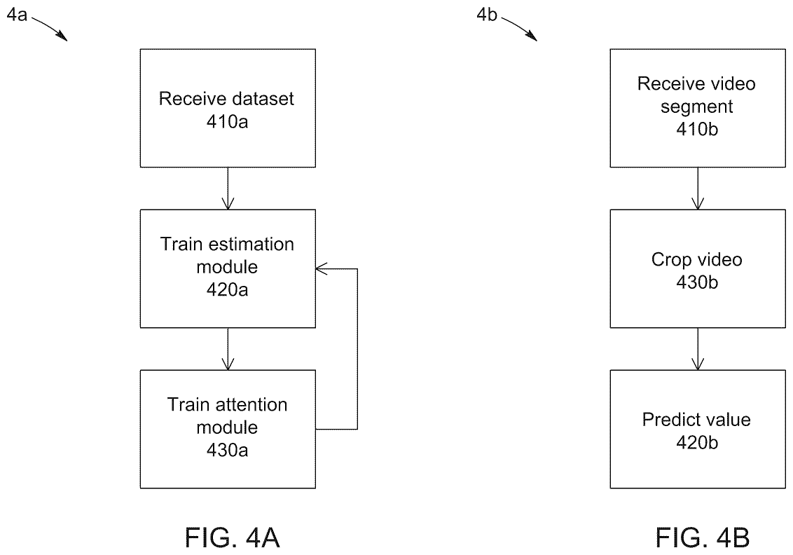

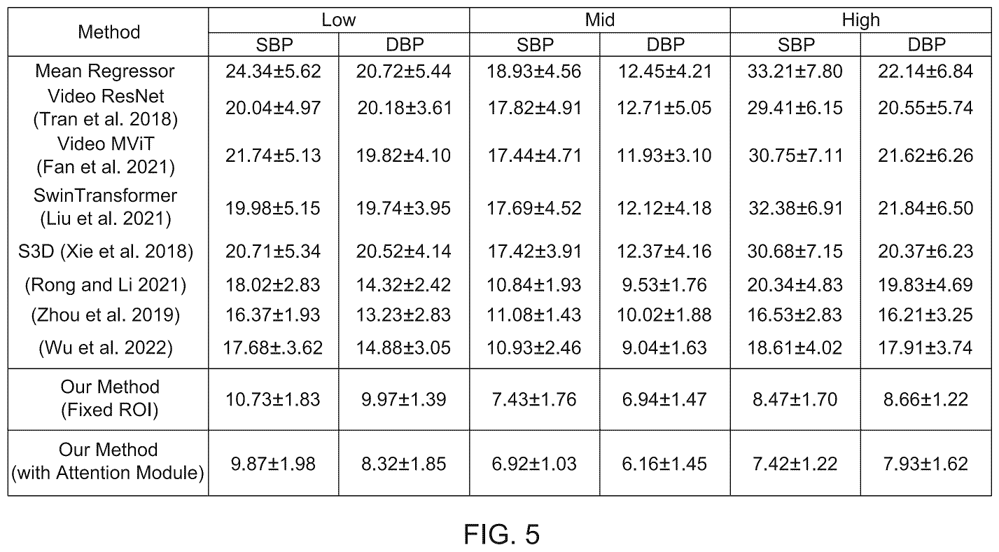

With reference to FIG. 4A, an exemplary method 4a for training a model to estimate vital features such as blood pressure values, a target physiological signal, is shown. Broadly described, method 4a includes receiving a training dataset, and successively training an estimation module and an attention module.

First step 410a includes receiving a training dataset. The dataset includes, for at least one training subject, a number of video segments and/or thermal images showing at least a skin exposed region of the subject such as the face of the subject, at least one first time series, each including measurements of a physiological parameter of the subject over the duration of the video segment, and at least one second time series, each including measurements of a vital feature value of the subject over the duration of the video segment. The first time series can for instance correspond to PPG, ECG, ICG and/or BCG measurements. The second time series can for instance correspond to SBP and/or DBP measurements. In some embodiments, one model is trained for one patient, and the only training subject is the targeted patient. In some embodiments, one model is trained for one patient, but the training dataset includes data from the targeted patient and data from additional training subjects. In some embodiments, one model is trained for a subset of patients or for all patients, and the dataset includes data from a number of training subjects. In some embodiments, one model is trained from a set of training subjects and then applied to a patient belonging to a different population, i.e., not included in the set of training subjects, such that the model can be used for zero-shot estimation of vital features of patients that were not used to train or calibrate the model.

Subsequent steps 420a and 430a can be performed in alternance for a number of times, for instance a configurable number, or over a configurable number of epochs, or until a predefined metric is verified with respect to the model performance. As the estimation module gets trained more, its performance in helping train the attention module will improve.

Step 420a includes training an estimation module including a neural network, for instance over a predefined number of epochs. The estimation module is configured to take as input a video segment, a cropped video segment, or patches of a video segment associated with an individual, and to predict and output an estimated value for one or more vital feature parameters, such as SBP and DPB.

The neural network of the estimation module can include a first block including an encoder, one or more second blocks including estimators for physiological parameters, and a third block including an estimator for estimating the vital features blood pressure. The first block can for instance be a convolutional encoder trained to accept a video segment, a cropped video segment, or patches of a video segment as input, and to generate a first vectorial embedding corresponding to a vectorial representation of the video content. Each of the second blocks can for instance include a predetermined number of fully connected layers trained to accept the first vectorial embedding as input, and to generate one of a number of second vectorial embeddings as output corresponding to a vectorial representation of a physiological parameter such as PPG, ECG, ICG and/or BCG. The third block can for instance include a predetermined number of fully connected layers trained to accept an aggregation of the second vectorial embedding such as a concatenation as input, and to generate estimated vital feature values such as SBP and/or DBP as output.

Training the estimation module includes training the neural network, i.e., optimizing parameters of the neural networks such as weights and/or biases in order to minimize a loss function that measures the amount of information lost between the actual, measured vital feature values of a training subject and the vital feature values estimated by the neural network. In some embodiments, the loss function is or includes a regression loss function, such as a mean squared error loss function. In some embodiments, the neural network is trained end-to-end, i.e., all blocks being trained at once. It can be appreciated that alternative means of training the neural network can be suitable. As an example, a subset of the blocks or one block can be trained at a time, with parameters of other blocks or a subset of these parameters being frozen.

In some embodiments, the neural network also includes one additional block for each physiological parameter. Each block can for instance include a convolutional encoder trained to accept a time series representing measured values of the physiological parameter over the duration of the video segment and/or features derives from these measured values, and to generate a vectorial embedding corresponding to a vectorial representation of the physiological parameter that is similar and/or comparable to the second vectorial embedding(s) generated by the second block(s) described above. In some of these embodiments, the loss function is or includes a contrastive loss function such as a symmetric cross entropy loss function allowing for video-physiological signal multimodal contrastive learning.

Step 430a includes training an attention module configured to extract patches of the video segment frames for inputting into the estimation module. Advantageously, the patches represent attentive regions of the frames including informative regions of the training subjects' faces. Training the attention module can include using a Monte Carlo policy gradient approach. As an example, training the attention module can be performed using the REINFORCE algorithm. In some embodiments, an agent, which can for instance be implemented as a trained machine learning model, is configured to perform an action at each of a number of steps. The actions performed can include different types of movement of the attentive region(s). The resulting attentive region(s) can then be used as input to the trained or partially trained estimation module to obtain an estimation of vital features of the training subjects. A reward based on the loss of the resulting estimations can then be attributed to the actions of the agent in order to reinforce beneficial actions.

With reference to FIG. 4B, an exemplary method 4b using a trained model to estimate vital features such as blood pressure values is shown. Method 4b includes, in a first step 410b, receiving a video showing at least a region of interest such as the face of a patient, in a subsequent step 430b, cropping the video by a trained attention module to obtain one or more patches corresponding to regions of the face that are predicted to be informative, and, in a final step 420b, estimating vital features such as SBP and DBP from the patches using an estimation module using a neural network trained to accept video patches as input and to generate estimated vital features as output.

To assess the performance of the approach described therein, extensive experiments were performed, using a uniquely recorded dataset. During training, both the physiological signals and video frames were used to train the model. However, during the test phase, only the video data was used, simulating real-world scenarios where only video information are available.

To evaluate the described framework, the only available dataset which contains video, PPG, ECG, ICB, blood pressure, all recorded in a synchronized manner, was used. The data is recorded from 12 subjects including both genders and from various nationalities, with different skin colours.

The PPG signal was recorded using a tiny ear clip connected to the participant's earlobe, and the ECG and ICG signals were recorded from electrodes placed on their skin. Finally, the participants wore a blood pressure measuring device on their right finger to measure the beat-to-beat BP.

The trial involved subjects sitting on a chair and engaging in various activities, including the Valsalva maneuver, cold pressor test and mental arithmetic tasks. These tasks are designed to induce both low and high blood pressure in participants, enabling the data to be used for estimating a wide range of BP values. The Valsalva maneuver involves inhaling air and then forcibly exhaling while closing the airways, resulting in increased intrathoracic pressure, leading to reduced venous return, ventricular filling, and cardiac output. The cold pressor test induces cold stress by subjecting participants to a sudden and progressively painful cold stimulus. This triggers a significant discharge of the sympathetic nervous system and the release of norepinephrine, causing various cardiovascular responses, such as arteriolar constriction, increased heart rate, and heightened cardiac contractility. These responses collectively lead to an increase in BP, known as the pressure response. Finally, in the arithmetic task, when the subjects are engaged in a mentally demanding activity, their bodies release stress hormones like adrenaline. This can result in a temporary increase in heart rate and blood pressure. These experiments ensure that a wide range of BP values and patterns exist in the dataset. In all our experiments, the objective is to estimate the systolictolic and diastolic peaks of blood pressure every T frames. The model disclosed herein was benchmarked against the following SOTA video processing and BP estimation baselines:

ResNet: ResNet, short for Residual Network, employs residual blocks, enabling the training of extremely deep networks without encountering the vanishing gradient problem.

MViT: MViT, or Multiscale Vision Transformer, combines the Vision Transformers (ViT) with a multiscale attention mechanism, enabling the model to process information at different spatial resolutions.

S3D: S3D, or Separable 3D Convolutional Networks, utilizes separable convolutions to reduce computational complexity.

SwinTransformer: SwinTransformer is a transformer-based architecture that adopts a hierarchical architecture with shifted windows to capture long-range dependencies efficiently.

Rong et al.: It is a facial-image-based BP measurement system that captures facial skin colour variations resulting from blood flow using a regular camera. The system extracts waveform features from these images and utilizes machine learning models, specifically multiple linear regression and support vector regression, to estimate BP. The approach focuses on leveraging the subtle changes in facial coloration due to pulsatile blood flow to predict BP non-invasively.

Zhou et al.: It is a noninvasive BP measurement method utilizing photoplethysmography (PPG) technology. This technique involves capturing skin colour changes correlated with heartbeats using a camera. The PPG signals are then processed to estimate BP. The system emphasizes the detection of minute colour variations in the skin induced by the cardiac cycle to infer BP readings. However, the method requires careful control of environmental factors such as light colour and intensity.

Wu et al.: It is a BP measurement system based on deep neural networks (DNN). This approach involves capturing facial signals and physiological indicators. They first extract PPG from video, and a deep neural network processes it along with other physiological signals to correlate them with BP readings. SBP and DBP values were categorized into high, low, and normal BP as follows: SBP in the [90, 130] range is normal (Mid), with values below 90 as Low and above 130 as High. DBP in the [60, 85] range is normal (Mid), with values below 60 as Low and above 85 as High. Finally, each group (Low, Mid, High) can be split into train/test sets using an 80/20 split, ensuring all SBP and DBP variations are represented in both train and test phases. The Mean Absolute Error (MAE) of each group, across subjects, will be used throughout our experiments.

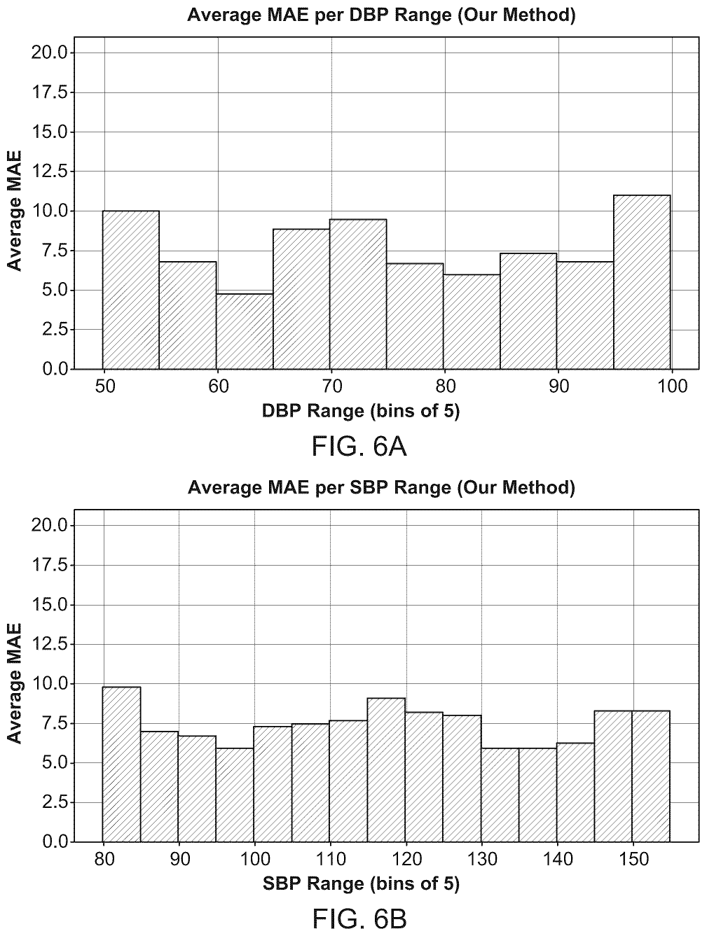

FIG. 5 shows the MAE (across all subjects) for low, mid, and high SBP and DBP values.

The comparison demonstrates that the disclosed method surpasses existing SOTA methods in accuracy for both SBP and DBP, particularly at the extremes of blood pressure values, which are critical for human health monitoring as accurate detection of high and low blood pressure is essential for proactive intervention. Notably, the performance gap is more pronounced for low and high blood pressure readings. The fact that most baseline models perform similarly to a mean regressor suggests a potential flaw in the evaluation protocol: many datasets predominantly feature data within the normal range, allowing models that predict near-normal values to perform well. Due to averaging, the impact of predictions outside the normal range is minimized since these cases are less frequent. Unlike other methods, the disclosed model achieves consistent performance across low, mid, and high blood pressure ranges, comparable to cuff-based devices.

The success of the disclosed method can be attributed to several innovations.

First, contrastive multimodal learning can be employed to refine video representations specifically for blood pressure estimation, integrating physiological signals during training. Unlike other SOTA methods that rely on pulse transit time, the disclosed end-to-end model achieves higher and more robust performance, particularly in non-normal blood pressure ranges.

Second, a balanced loss function can be used to address blood pressure value imbalance during training. This approach ensures consistent performance for high and low blood pressure values, preventing the prevalence of normal-range values from diminishing the impact of non-normal values on the cost function.

Finally, an attention module that focuses on the most relevant facial regions can be incorporated. A version of the disclosed framework with three fixed ROIs—on the forehead and each cheek—was also tested. Even without the attention module i.e., with fixed ROIs, the disclosed model significantly outperforms SOTA methods. The attention module can further improve performance by dynamically adapting to the best-quality facial regions for blood pressure estimation. This feature is crucial for scenarios with occlusions or varying lighting conditions, which can impair fixed-ROI models.

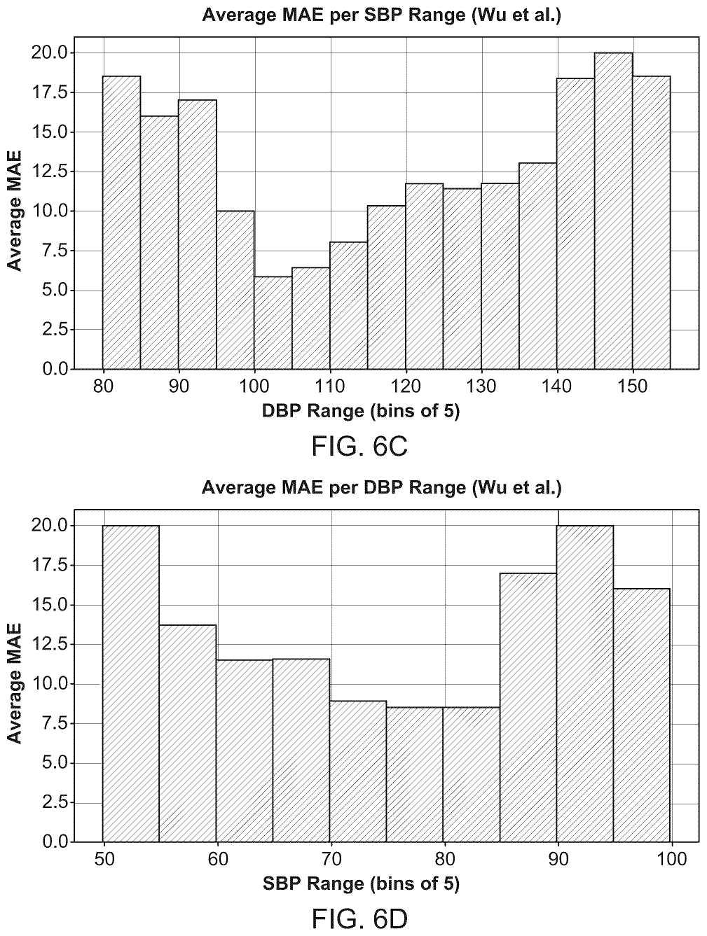

The distribution of SBP and DBP values is centred around the normal range, making it difficult to assess algorithm performance accurately. The results show that the disclosed model consistently performs well across normal, low, and high blood pressure ranges. FIGS. 6A, 6B, 6C, and 6D compare the MAEs for SBP and DBP across subjects in 5-unit bins with Wu, B.-F.; Wu, B.-J.; Tsai, B.-R.; and Hsu, C.-P: A Facial-Image-Based Blood Pressure Measurement System Without Calibration. In IEEE Transactions on Instrumentation and Measurement (2022). The disclosed model maintains nearly uniform error levels across all ranges, unlike Wu et al., and effectively generalizes to low and high blood pressure detection. This balanced performance highlights the robustness and versatility of the disclosed approach in real-world scenarios.

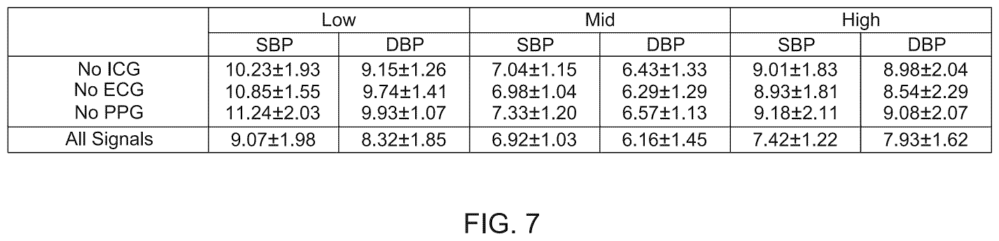

To assess the impact of each component on the model's performance, they were systematically removed one at a time and the results were compared with the complete model. Specifically, λ₁=0, λ₂=0, and λ₁+λ₂=1 can be set to exclude PPG, ECG, and ICG influences during training. Results, shown in FIG. 7, reveal that including all signals yields the best performance. Removing any signal does not significantly degrade overall performance, maintaining consistency across BP values and still outperforming the SOTA. However, each removed signal negatively impacts performance, indicating that while all signals are beneficial, the model is not overly dependent on any single one. This balance allows the model to perform well even if one signal is noisy or missing, enhancing adaptability and robustness in real-world scenarios.

Notably, the impact of removing signals is more pronounced in the low and high BP ranges, significantly affecting performance in these ranges. This highlights that incorporating physiological signals during training improves performance and addresses BP value imbalance, helping the model better identify patterns associated with low and high BP for more accurate predictions.

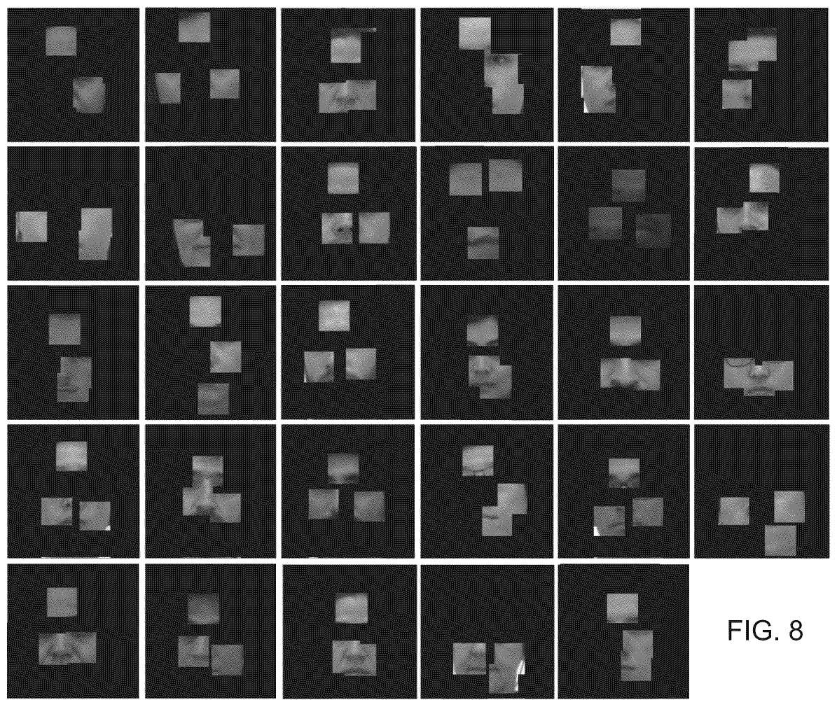

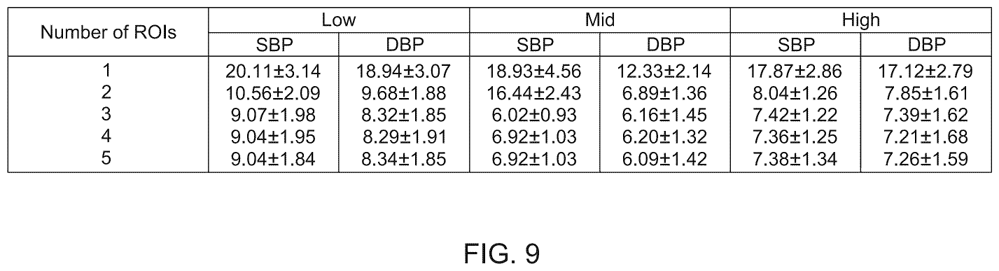

To understand what the Attention module learns, the identified ROIs are visualized for all 29 subjects in FIG. 8, using randomly selected frames. As can be seen, these ROIs are near the cheeks, lips, and forehead. While cheeks and forehead are known BP extraction sites, variations like gender, skin type, accessories, or facial hair may affect this. The proposed Attention Module accounts for these variations by dynamically focusing on unobstructed facial areas, improving accuracy. The number of regions the Attention Module selects were tested, from 1 to 5, as shown in FIG. 9. Results reveal that using a single region leads to significant error, indicating insufficient information from one ROI. Increasing to three regions optimizes performance, while more regions add complexity without substantial gains in accuracy. Thus, three regions offer the best balance of accuracy and efficiency.

An interesting experiment is to explore whether including data from other subjects can improve estimation performance for a specific subject. This leads to two important research questions: (1) Can performance be improved by using a model pre-trained on data from all other subjects? (2) Can this model perform zero-shot inference, estimating blood pressure (BP) for a new subject without any training on the new subject?

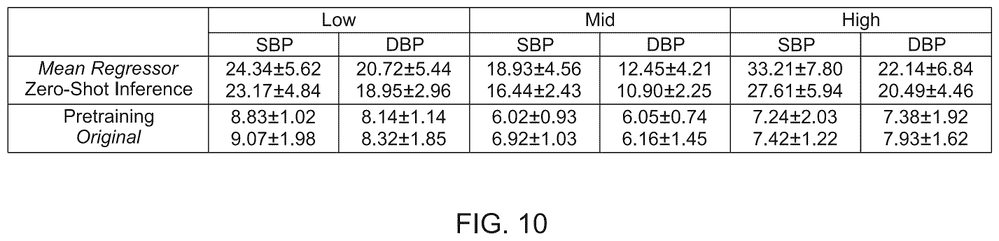

To address these, the model was trained on data from all subjects except one and evaluated its performance in both scenarios, as shown in FIG. 10. Results show that pre-training enhances performance, especially for mid-range BP values, with noticeable improvements for low and high values as well. For zero-shot inference, performance drops significantly but still outperforms the mean regressor. This drop is expected, as even advanced BP monitoring devices typically require calibration. These results suggest that calibration-free video-based BP estimation frameworks warrant further investigation. With more data, models might achieve performance improvements akin to those seen in Large Language Models, where extensive pre-training greatly enhances results. Overall, the experiment underscores the potential for further improvement with additional subjects.

The present disclosure makes it possible to rely solely on video data during the inference phase for blood pressure assessment. Unlike the cuff-based methods, it is capable of providing a real-time continuous beat-to-beat BP estimation. Incorporating physiological signals during training, and discarding them in the test phase, is a groundbreaking approach employed for the first time. The present disclosure also demonstrates the possibility of applying multimodal contrastive learning (MMCL) beyond traditional domains such as vision, text, or audio. The integration of physiological signals with video demonstrates the potential of MMCL in a new context, which is video and biosignals. The present disclosure also demonstrates that integrating RL-based attention finding in our model for detecting the best regions of interest in the face for BP estimation provides an advantageous approach for telehealth-based monitoring. The effectiveness of the disclosed approach is demonstrated through extensive experiments on a unique dataset of physiological signals and video that includes a wide range of BP from low to high on diverse human subjects with different skin colour, gender, and race. The results demonstrate a robust performance for BP estimation using the disclosed systems and methods, by achieving mean squared errors as low as 0.05.

For the sake of simplicity, the above description focuses on an embodiment of the invention that predicts systolic blood pressure (SBP) and diastolic blood pressure (DBP) as the two vital features. However, it should be appreciated that the disclosed systems and methods can readily be extended to include additional physiological signals such as electrocardiogram (ECG), impedance cardiography (ICG), photoplethysmography (PPG), respiration rate, heart rate, and heart rate variability. Training for additional or distinct physiological signals can for instance occur at the same time, in parallel, via different cost functions and encoders, each optimized for a specific vital sign. Once the multidimensional database is acquired and used for training the networks and/or the encoders during the training phase, the inference phase can be performed as “zero-shot”, that is, recalibration and/or retraining for the users may not be required. In some embodiments, this can be performed with one video stream only. In some embodiments, a thermal camera can additionally or alternatively be used.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative and non-limiting and it will be understood by persons skilled in the art that other variants and modifications may be made without departing from the scope of the invention as defined in the claims appended hereto.

1. A computer-implemented method for estimating at least one vital feature of a patient, the method comprising:
receiving a video segment showing at least one skin-exposed region of the patient;
cropping the video segment to create at least one video patch; and
generating the at least one estimated vital feature of the patient by providing the at least one video patch as input to a neural network trained on a training dataset comprising a plurality of training video segments of a plurality of training subjects, each training video segment showing at least one skin-exposed region of a training subject of the plurality of training subjects and being associated with at least one first time series corresponding to values of a measured physiological signal of the training subject and at least one second time series corresponding to measured values of a vital feature of the training subject,
wherein the neural network is configured to generate a representation of at least one-time series of predicted values of a predicted physiological signal of the patient and to generate the at least one estimated vital feature based on the representation, and
wherein the patient and the plurality of training subjects belong to different populations.

2. The method of claim 1, further comprising receiving and cropping a thermal image of the patient, wherein the training dataset further comprises a plurality of training images of the plurality of training subjects.

3. The method of claim 1, wherein the neural network comprises:
a neural network-based encoder trained to accept the at least one video patch as input and to generate a first vectorial embedding as output;
at least one fully connected block trained to accept the first vectorial embedding as input and to generate at least one second vectorial embedding as output corresponding to the representation of the time series of predicted values of the predicted physiological signal of the patient; and
at least one fully connected block trained to accept an aggregation of the at least one second vectorial embedding and to generate the at least one estimated vital feature.

4. The method of claim 1, wherein cropping the video segment comprises using an attention module trained using reinforcement learning based on a reward corresponding to an error of the neural network in predicting the measured values of the vital feature of the training subject.

5. A system for estimating at least one vital feature of a patient from at least a video segment showing at least one skin-exposed region of the patient, the system comprising at least one memory for storing parameters of a neural network, the neural network comprising:
an encoder trained to accept at least one video patch of the video segment as input and to generate a first vectorial embedding as output;
at least one fully connected block trained to accept at least the first vectorial embedding as input and to generate at least one second vectorial embedding as output corresponding to a representation of a time series of predicted values of a physiological signal of the patient; and
at least one fully connected block trained to accept an aggregation of the at least one second vectorial embedding and to generate the at least one estimated vital feature for the patient.

6. The system of claim 5, wherein the estimation is performed further from a thermal image of the patient, an additional encoder being trained to accept the thermal image of the patient as input and to generate an additional first vectorial embedding as output, the at least one fully connected block trained to accept further the additional first vectorial embedding as input.

7. The system of claim 5, wherein the at least one fully connected block is trained to accept multiple vital-relevant input modalities and output multiple vital features.

8. The system of claim 5, wherein the physiological signal is one of a photoplethysmography signal, an electrocardiography signal, a ballistocardiography signal, and an impedance cardiography signal.

9. The system of claim 5, wherein the memory further stores a training dataset comprising a plurality of training video segments, each training video segment showing at least one skin-exposed region of a training subject from a set of at least one training subject and being associated with at least one first time series corresponding to values of a measured physiological signal of the training subject and at least one second time series corresponding to measured values of a vital feature of the training subject, and further comprising at least one processor configured for training the neural network on the training dataset.

10. The system of claim 9, wherein the set of at least one training subject consists of the patient.

11. The system of claim 9, wherein the patient and the set of at least one training subject belong to different populations.

12. The system of claim 5, wherein the neural network is trained using an aggregation of a plurality of loss functions.

13. The system of claim 12, wherein one of the loss functions is an aggregation of at least one contrastive loss function based on comparing the at least one second vectorial embedding and corresponding at least one vectorial embedding of each measured physiological signal.

14. The system of claim 12, wherein one of the loss functions is an aggregation of at least one error function based on comparing the at least one vital feature and corresponding at least one measured vital feature.

15. The system of claim 5, wherein the at least one memory further stores parameters of an attention module trained to crop the at least one video patch from the video segment.

16. The system of claim 15, further comprising at least one processor configured to train the attention module using reinforcement learning based on a reward corresponding to an error of the neural network in predicting measured values of the vital feature of a set of at least one training subject.

17. The system of claim 15, wherein the attention module is trained to crop at least two video patches from the video segment.

18. The system of claim 15, further comprising at least one processor configured to train the neural network and the attention module by repeating a first and a second training stages, wherein:
in the first training stage, the parameters of the attention module are frozen and the parameters of the neural network are optimized; and
in the second training stage, the parameters of the neural network are frozen and the parameters of the attention module are optimized.

19. The system of claim 5, wherein the at least one estimated vital feature comprises an estimated beat-to-beat blood pressure.

20. A non-transitory computer-readable medium having instructions stored thereon which, when executed by one or more processors, cause the one or more processors to perform a method comprising:
receiving a video segment showing at least one skin-exposed region of a patient;
cropping the video segment to create at least one video patch; and
generating at least one estimated vital feature of the patient by providing the at least one video patch as input to a neural network trained on a training dataset comprising a plurality of training video segments of a plurality of training subjects, each training video segment showing at least one skin-exposed region of a training subject of the plurality of training subjects and being associated with at least one first time series corresponding to values of a measured physiological signal of the training subject and at least one second time series corresponding to measured values of a vital feature of the training subject,
wherein the neural network is configured to generate a representation of at least one-time series of predicted values of a predicted physiological signal of the patient and to generate the at least one estimated vital feature based on the representation, and
wherein the patient and the plurality of training subjects belong to different populations.
